(19) United States
(12) Patent Application Publication
(10) Pub. No.: US 2025/0107883 A1
(43) Pub. Date: Apr. 3, 2025

(54) INTERFERENCE SCREWS

(71) Applicant: ARTHREX, INC., Naples, FL (US)

(72) Inventors: Peter J. DREYFUSS, Naples, FL (US); Kyle F. CHUN, Chino Valley, AZ (US)

(21) Appl. No.: 18/419,707

(22) Filed: Jan. 23, 2024

Related U.S. Application Data
(60) Provisional application No. 63/586,066, filed on Sep. 28, 2023.

(51) Int. Cl. A61F 2/08

(52) U.S. Cl. CPC A61F 2/0811; A61F 2002/0829; A61F 2002/0858; A61F 2220/0033

(57) ABSTRACT

Graft fixation systems and methods are provided for securing a graft within a bone tunnel. An exemplary graft fixation system may include an interference screw adapted for fixating both a graft and a reinforcement suture relative to bone.

### CROSS-REFERENCE TO RELATED APPLICATIONS

This application claims the benefit of U.S. Provisional Application No. 63/586,066, which was filed on Sep. 28, 2023 and is incorporated herein by reference in its entirety.

### BACKGROUND

This disclosure relates to the field of surgery, and more particularly to graft fixation systems that include an interference screw adapted to both externally fixate a graft and internally fixate a reinforcement suture that is separate from the graft.

Tissue reconstruction surgeries, such as anterior cruciate ligament (ACL) reconstructions and posterior cruciate ligament (PCL) reconstructions, typically involve drilling a tunnel through bone, positioning a substitute graft in the bone tunnel, and fixating the graft within the bone tunnel using one or more fixation devices, such as buttons, screws, or the like.

### SUMMARY

This disclosure relates to graft fixation systems and methods for securing a graft within a bone tunnel. The graft fixation systems may include a surgical button, a loop connected to the surgical button, a graft carried by the loop, a reinforcement suture connected to the button and tensionable separately from the graft, and an interference screw. The graft fixation system can be used in various tissue reconstruction procedures, including but not limited to, ACL and PCL reconstructions, for example.

An exemplary interference screw may include, inter alia, a body including an outer diameter wall that includes a thread and an inner diameter wall that circumscribes a cannulation. The thread is configured to engage a bone to trap a graft between the body and the bone, and a locking barb is provided within the cannulation and is configured to lock a reinforcement suture relative to the bone.

An exemplary surgical method may include, inter alia, loading a reinforcement suture through a cannulation of an interference screw, inserting the interference screw within a bone tunnel of a bone, thereby trapping a graft between the bone and the interference screw, tensioning the reinforcement suture in a first direction, and locking the reinforcement suture within the cannulation to prevent movement in a second direction.

Another exemplary surgical method may include, inter alia, inserting an interference screw into a bone tunnel of a bone. The interference screw externally fixates a graft relative to the bone and internally locks a reinforcement suture relative to the bone.

The embodiments, examples, and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates a graft fixation system for securing a graft within a bone tunnel.

FIG. 2 illustrates an exemplary interference screw of the graft fixation system of FIG. 1.

FIG. 3 is a cross-sectional view through section 3-3 of FIG. 2.

FIG. 4A is a blown-up view of region 4 of FIG. 3.

FIG. 4B illustrates a plurality of rows of locking barbs of the interference screw of FIG. 2.

FIG. 5 schematically illustrates an exemplary use of the interference screw of FIGS. 2-4 for externally fixating a graft and internally fixating a reinforcement suture.

FIGS. 6 and 7 illustrate another exemplary interference screw for a graft fixation system.

FIG. 8 illustrates an exemplary reinforcement suture of a graft fixation system.

### DETAILED DESCRIPTION

This disclosure relates to graft fixation systems and methods for securing a graft within a bone tunnel. The graft fixation systems may include a surgical button, a loop connected to the surgical button, a graft carried by the loop, a reinforcement suture connected to the button and tensionable separately from the graft, and an interference screw. The graft fixation system can be used in various tissue reconstruction procedures, including but not limited to, ACL and PCL reconstructions, for example. These and other features of this disclosure are described in further detail below.

An exemplary interference screw may include, inter alia, a body including an outer diameter wall that includes a thread and an inner diameter wall that circumscribes a cannulation. The thread is configured to engage a bone to trap a graft between the body and the bone, and a locking barb is provided within the cannulation and is configured to lock a reinforcement suture relative to the bone.

In any further embodiment, the locking barb is an integral feature of the body.

In any further embodiment, the locking barb is part of a locking ferrule that is received within the cannulation.

In any further embodiment, the locking ferrule is secured to the body via an interference fit.

In any further embodiment, the locking ferrule is secured to the body via an overmolding.

In any further embodiment, the locking barb includes a pointed tip.

In any further embodiment, the body extends along a longitudinal axis between a drive head located at a proximal end and a tip located at a distal end.

In any further embodiment, the locking barb is angled in a direction toward the drive head.

In any further embodiment, the locking barb is located closer to the tip than the drive head.

In any further embodiment, a plurality of locking barbs protrude inwardly from the inner diameter wall.

An exemplary surgical method may include, inter alia, loading a reinforcement suture through a cannulation of an interference screw, inserting the interference screw within a bone tunnel of a bone, thereby trapping a graft between the bone and the interference screw, tensioning the reinforcement suture in a first direction, and locking the reinforcement suture within the cannulation to prevent movement in a second direction

In any further embodiment, a thread of the interference screw external fixates the graft.

In any further embodiment, a locking barb of the interference screw internally locks the reinforcement suture.

In any further embodiment, a locking barb protrudes inwardly from an inner diameter wall of the interference screw.

In any further embodiment, the locking barb is part of a locking ferrule that is received within the cannulation.

In any further embodiment, the surgical method further includes inserting a locking ferrule into the cannulation prior to loading and tensioning the reinforcement suture.

In any further embodiment, the locking barb engages a thickened section of the reinforcement suture to prevent its movement in the second direction.

Another exemplary surgical method may include, inter alia, inserting an interference screw into a bone tunnel of a bone. The interference screw externally fixates a graft relative to the bone and internally locks a reinforcement suture relative to the bone.

In any further embodiment, a thread of the interference screw externally fixates the graft.

In any further embodiment, a locking barb of the interference screw internally fixates the reinforcement suture.

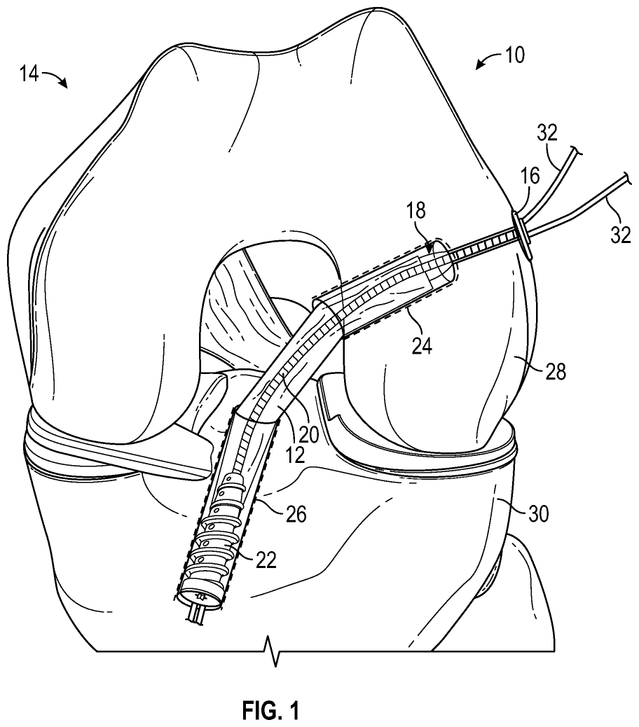

FIG. 1 illustrates an exemplary graft fixation system 10 for fixating a graft 12 within a joint 14 (e.g., a knee joint). The graft fixation system 10 may be used to fixate the graft 12 when performing a variety of tissue reconstruction procedures. The tissue reconstruction procedures could include any surgical procedure in which it is desirable to position a replacement graft within a bone tunnel to repair torn tissue. ACL and PCL reconstructions are but two non-limiting examples of surgical reconstruction procedures that could benefit from the use of the graft fixation systems 10 of this disclosure.

The graft fixation system 10 may be implanted within the joint 14 to repair a torn tissue (e.g., a torn ACL). Prior to implanting the graft fixation system 10 within the joint 14, a first bone tunnel 24 (e.g., a socket) may be formed in a first bone 28 (e.g., a femur), and a second bone tunnel 26 (e.g., a passage) may be formed in a second bone 30 (e.g., a tibia).

The graft fixation system 10 may include, among various other components or features, a surgical button 16, a loop 18, a reinforcement suture 20, and an interference screw 22. The surgical button 16 may be configured to provide cortical bone fixation of the graft 12 relative to the first bone 28 after the graft 12 has been positioned within the first bone tunnel 24. The surgical button 16 may include one or more apertures formed through the body of the surgical button 16 for receiving and carrying both the loop 18 and the reinforcement suture 20. The surgical button 16 may be oblong or round and may be made of either metallic or polymeric materials within the scope of this disclosure.

The loop 18 may be an adjustable loop made of a flexible material (e.g., suture) and includes an adjustable length and perimeter. Tensioning strands 32 of the loop 18 may be pulled to reduce the size of the loop 18. For example, the loop 18 may be reduced in size by pulling the tensioning stands 32 in a first direction but is prevented from loosening in the opposite direction due to applied internal tensile forces. The tensioning strands 32 may be spliced through portions of the loop 18 for establishing splices that are configured to constrict when tensioned to create the applied internal tensile forces.

The graft 12 and the reinforcement suture 20 may be connected at different locations of the graft fixation system 10. For example, the graft 12 may be connected to the loop 18, and the reinforcement suture 20 may be connected to the surgical button 16. The graft 12 may be looped over a cradle portion of the loop 18, and the reinforcement suture 20 may be received through apertures formed through the surgical button 16. The reinforcement suture 20 is therefore unconnected to the graft 12 and may be tensioned separately from the graft 12.

The graft 12 and the reinforcement suture 20 can be tensioned separately from one another, resulting in independent tension loads. Tension can also be reapplied to the graft 12 after tensioning the reinforcement suture 20. Accordingly, joint loads may be shared between the graft 12 and the reinforcement suture 20, with the reinforcement suture 20 acting as a dynamic joint stabilizer that shares loads with and reinforces the primary repair provided by the graft 12. The reinforcement suture 20 may therefore be referred to as an “internal brace” or “safety belt” feature of the graft fixation system 10.

The reinforcement suture 20 may include one or more strands of suture tape, such as FiberTape®. FiberTape® is a suture product marketed and sold by Arthrex, Inc. However, other suture products could be utilized for the reinforcement suture 20 within the scope of this disclosure.

The reinforcement suture 20 may include a varying thickness. The reinforcement suture 20 may therefore include one or more tapered regions 58 where the reinforcement suture 20 transitions between a thickened section 60 and a thinned section 62 (see, e.g., FIG. 8). The thinned sections 62 can facilitate passing the reinforcement suture 20 through the cannulation 44, such as via a suitable suture loader that can be provided as part of a suture locking system that includes the interference screw 22, for example, and the thickened sections 60 can provide greater surface area for the locking barbs 52 to engage in order to sufficiently lock the reinforcement suture 20 relative to the interference screw 22.

In an embodiment, the thickened sections 60 of the reinforcement suture 20 are about twice as thick as the thinned sections 62. However, other ratios (e.g., 1.5:1, 3:1, etc.) between the relative thicknesses (e.g., outer diameters) of the thickened sections 60 and the thinned sections 62 are contemplated within the scope of this disclosure.

In an embodiment, the graft fixation system 10 may be implanted within the joint 14 by passing the surgical button 16 through the first bone tunnel 24. The surgical button 16 may be pulled through the first bone tunnel 24 using a passing suture (not shown) and can self-flip onto the cortex of the first bone 28 once tension is released on the passing suture. Alternatively, a flipping suture (not shown) could be used to seat the surgical button 16 relative to the cortex.

After passing and flipping the surgical button 16, the loop 18 may suspend the graft 12 within the first bone tunnel 24. The tensioning strands 32 can be pulled to adjust the size of the loop 18 and to fully seat the graft 12 within the first bone tunnel 24.

Both the graft 12 and the reinforcement suture 20 may subsequently be arranged to extend within the second bone tunnel 26 and can be separately tensioned prior to completing the technique. For example, a first tension can be applied to the graft 12 (such as by applying traction to a suture that is whipstitched to the graft 12), and a second, different tension can be applied to the reinforcement suture 20 since these components are unconnected to one another. The graft 12 can thus be retensioned after intraoperative preconditioning. Intraoperative preconditioning can be used to reduce residual laxity in the graft 12. Graft retensioning optimizes the mechanical stability of soft tissue by decreasing its dynamic elongation. The reinforcement suture 20 (e.g., FiberTape®) provides an increased stiffness compared to soft tissue material, thus its resistance to dynamic elongation may be high enough without retensioning.

The graft 12 and the reinforcement suture 20 can both be fixated relative to the second bone 30 by the interference screw 22. In prior techniques, a separate screw or anchor was necessary to fixate the reinforcement suture 20 when using interference screw fixation to secure the graft 12 relative to the second bone 30. However, as further discussed below, the interference screw 22 may be specifically configured to both externally fixate the graft 12 (e.g., by trapping it between the interference screw 22 and the second bone tunnel 26) and internally fixate the reinforcement suture 20 (e.g., via a one-way locking mechanism of the interference screw 22).

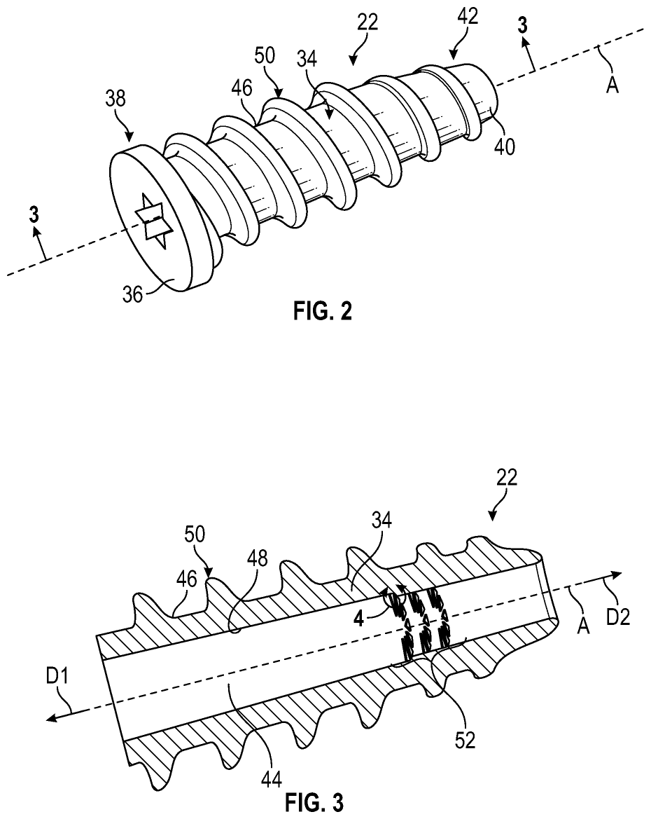

FIGS. 2 and 3 illustrate an exemplary interference screw 22 that can be utilized as part of the graft fixation system 10 described above. The interference screw 22 may include a body 34 that extends along a longitudinal axis A between a drive head 36, provided at a proximal end 38, and a tip 40, provided at a distal end 42. The body 34 may or may not taper between the drive head 36 and the tip 40. The specific size and shape of the body 34 are not intended to limit this disclosure.

The drive head 36 may be configured for accommodating a driver for insertion of the interference screw 22 into bone. The drive head 36 may include a hex opening or any other shaped opening for receiving a correspondingly shaped tip of a driver (not shown).

The tip 40 may be shaped in any manner to facilitate insertion of the interference screw 22 into bone. For example, the tip 40 may be blunted, rounded, pointed, or otherwise shaped.

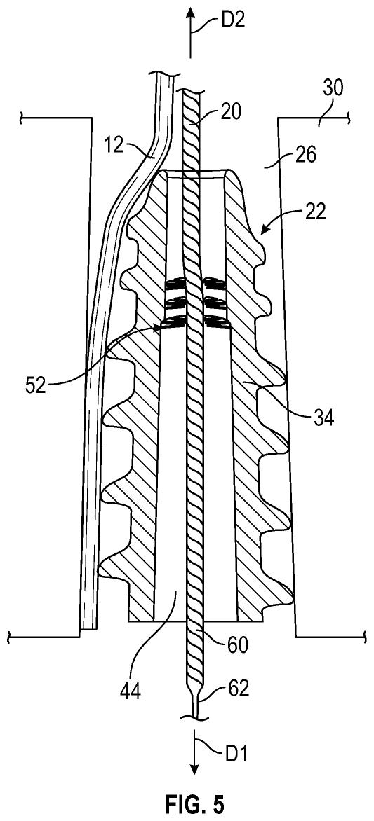

A cannulation 44 (see FIG. 3) may extend through the body 34 and may establish an internal passageway for accommodating the reinforcement suture 20 (see, for example, FIG. 5) or other structures (e.g., wires, shafts, etc.). The cannulation 44 may extend across the entire length of the body 34 and thus extends from the tip 40 to the drive head 36. The longitudinal axis A may bisect the cannulation 44.

The body 34 may include an outer diameter wall 46. A thread 50 may wrap around the outer diameter wall 46, such as in a helical pattern. The thread 50 may extend substantially from the proximal end 38 to the distal end 42 of the body 34. The thread 50 may be a continuous or a discontinuous thread that is configured to engage bone as the interference screw 22 is inserted into a bone tunnel. The various designs characteristics of the thread 50, including thread pitch, thread angle, etc., are not intended to limit this disclosure.

The body 34 may additionally include an inner diameter wall 48 (see FIG. 3). The inner diameter wall 48 may circumscribe the cannulation 44. In some implementations, the cannulation 44 tapers in a direction toward the distal end 42 and is therefore narrower within the tip 40 compared to within the drive head 36.

A plurality of locking barbs 52 may protrude inwardly from the inner diameter wall 48. The locking barbs 52 may therefore occupy at least a portion of the open space of the cannulation 44. In an embodiment, the locking barbs 52 are integrally formed (e.g., molded) features of the body 34. The locking barbs 52 may be located closer to the distal end 42 than to the proximal end 38. However, the locking barbs 52 could be located anywhere along the length of the cannulation 44. The locking barbs 52 may be either rigid or flexible structures.

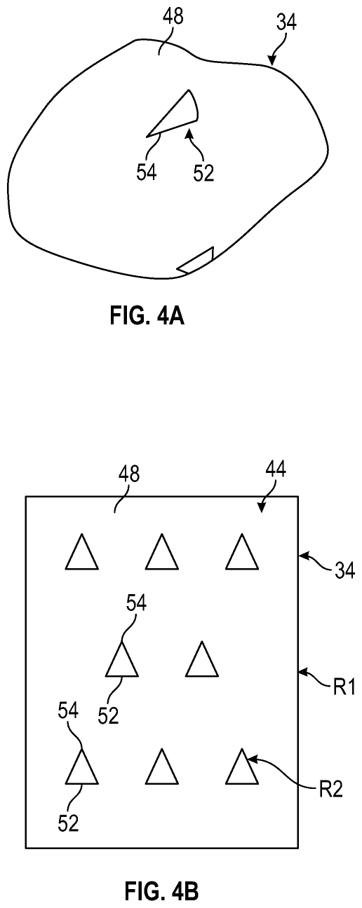

Each locking barb 52 may include a sharp or pointed tip 54 (see FIG. 4A), and each locking barb 52 may be angled in a direction toward the proximal end 38 and thus the drive head 36. The locking barbs 52 may therefore establish a one-way locking mechanism that permits one or more structures, such as the reinforcement suture 20, to pass through the cannulation 44 in a first direction D1 while preventing the structure from being tensioned or otherwise moved in a second direction D2.

The locking barbs 52 may be arranged in multiple rows along the length of the cannulation 44. For example, the locking barbs 52 may be arranged in a least a first row R1 and a second row R2 (see FIG. 4B). In an embodiment, the locking barbs 52 of the second row R2 are staggered relative to the locking barbs 52 of the first row R1 (see FIG. 4B).

Referring now to FIG. 5, with continued reference to FIGS. 1-4, the interference screw 22 may be inserted into the second bone tunnel 26, thereby trapping the graft 12 (via interference) between the body 34 of the interference screw 22 and the second bone 30. The reinforcement suture 20 may be received through the cannulation 44 prior to inserting the interference screw 22 into the second bone tunnel 26. For example, one or more thinned sections 62 of the reinforcement suture 20 may be passed through the cannulation 44 by pulling the thinned sections 62 in the first direction D1.

Once the reinforcement suture 20 has been passed through the cannulation 44 and the interference screw 22 has been inserted into the bone tunnel 26, the reinforcement suture 20 may be tensioned in the first direction D1 to lock the reinforcement suture 20 relative to the second bone 30. The locking barbs 52 prevent the reinforcement suture 20 from backing up or otherwise moving in the second direction D2. For example, if tension is applied in the second direction D2, the pointed tips 54 of the locking barbs 52 may interdigitate with one or more of the thickened sections 60 of the reinforcement suture 20 and thereby prevent it from moving in the second direction D2. The interference screw 22 is therefore equipped to provide the dual function of externally fixating the graft 12 and internally fixating the reinforcement suture 20 relative to the second bone 30.

Although shown as locking a single reinforcement suture 20 in the above implementations, the interference screw 22 could be configured to receive and lock multiple strands of suture.

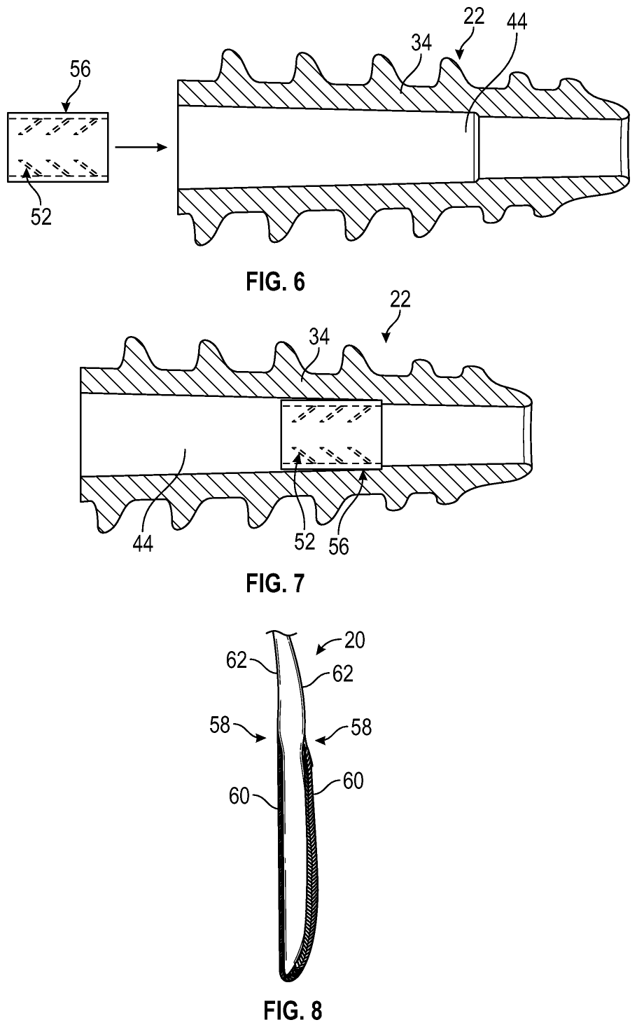

In the above embodiment, the locking barbs 52 are integral features of the body 34 of the interference screw 22. However, other implementations are contemplated within the scope of this disclosure. For example, as shown in FIGS. 6-7, the locking barbs 52 could be integral features of a separate locking ferrule 56 that is insertable into the cannulation 44 of the interference screw 22. The locking ferrule 56 may be secured to the body 34 of the interference screw 22 via an interference fit, for example. The locking ferrule 56 can be accommodated within the cannulation 44 either during manufacturing (e.g., via an overmolding process) or while performing the surgical reconstruction procedure (such as, for example, just prior to inserting the interference screw 22 into a bone tunnel). Inserting the locking ferrule 56 during the surgical procedure can simplify suture handling and prevent the premature locking of the reinforcement suture 20.

The graft fixation systems and surgical methods described herein may be utilized to secure grafts within bone tunnels. The graft fixation systems may include interference screws that include internal one-way locking mechanisms. Accordingly, a single interference screw can be utilized for achieving external fixation of a graft and internal fixation of a reinforcement suture.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

What is claimed is:

1. An interference screw, comprising:
a body including an outer diameter wall that includes a thread and an inner diameter wall that circumscribes a cannulation;
the thread configured to engage a bone to trap a graft between the body and the bone; and
a locking barb extending from the inner diameter wall and configured to lock a reinforcement suture relative to the bone.

2. The interference screw as recited in claim 1, wherein the locking barb is an integral feature of the body.

3. The interference screw as recited in claim 1, wherein the locking barb is part of a locking ferrule that is received within the cannulation.

4. The interference screw as recited in claim 3, wherein the locking ferrule is secured to the body via an interference fit.

5. The interference screw as recited in claim 3, wherein the locking ferrule is secured to the body via an overmolding.

6. The interference screw as recited in claim 1, wherein the locking barb includes a pointed tip.

7. The interference screw as recited in claim 1, wherein the body extends along a longitudinal axis between a drive head located at a proximal end and a tip located at a distal end.

8. The interference screw as recited in claim 7, wherein the locking barb is angled in a direction toward the drive head.

9. The interference screw as recited in claim 7, wherein the locking barb is located closer to the tip than the drive head.

10. The interference screw as recited in claim 1, comprising a plurality of locking barbs protruding inwardly from the inner diameter wall.

11. A surgical method, comprising:
loading a reinforcement suture through a cannulation of an interference screw;
inserting the interference screw within a bone tunnel of a bone,
wherein inserting the interference screw traps a graft between the bone and the interference screw;
tensioning the reinforcement suture in a first direction; and
locking the reinforcement suture within the cannulation to prevent movement in a second direction.

12. The surgical method as recited in claim 11, wherein a thread of the interference screw external fixates the graft.

13. The surgical method as recited in claim 11, wherein a locking barb of the interference screw internally locks the reinforcement suture.

14. The surgical method as recited in claim 13, wherein the locking barb protrudes inwardly from an inner diameter wall of the interference screw.

15. The surgical method as recited in claim 13, wherein the locking barb is part of a locking ferrule that is received within the cannulation.

16. The surgical method as recited in claim 15, comprising:
inserting the locking ferrule into the cannulation prior to loading and tensioning the reinforcement suture.

17. The surgical method as recited in claim 13, wherein the locking barb engages a thickened section of the reinforcement suture to prevent its movement in the second direction.

18. A surgical method, comprising:
inserting an interference screw into a bone tunnel of a bone,
wherein the interference screw externally fixates a graft relative to the bone and internally locks a reinforcement suture relative to the bone.

19. The surgical method as recited in claim 18, wherein a thread of the interference screw externally fixates the graft.

20. The surgical method as recited in claim 18, wherein a locking barb of the interference screw internally fixates the reinforcement suture.
